(19) United States
(12) Patent Application Publication
(10) Pub. No.: US 2024/0024395 A1
(43) Pub. Date: Jan. 25, 2024

(54) CONJUGATED T-2 TOXIN TO PROTECT AGAINST MYCOTOXICOSIS

(71) Applicant: INTERVET INC., Madison, NJ (US)

(72) Inventors: Sietske Kooijman, Huissen (NL); Ruud Philip Antoon Maria Segers, Boxmeer (NL); Maarten Hendrik Witvliet, Oostrum (NL)

(73) Assignee: INTERVET INC., Madison, NJ (US)

(21) Appl. No.: 18/257,420

(22) PCT Filed: Dec. 21, 2021
(86) PCT No.: PCT/EP2021/086950

§ 371 Date: Jun. 14, 2023

(30) Foreign Application Priority Data
Dec. 22, 2020 (EP) 20216338.2

(51) Int. Cl. A61K 36/06; A61K 31/343; A61K 47/64; A61P 39/02

(52) U.S. Cl. CPC A61K 36/06; A61K 31/343; A61K 47/643; A61K 47/646; A61P 39/02; A61K 2039/552

(57) ABSTRACT

The present invention pertains to the use of conjugated T-2 toxin (T2) in a method to protect an animal against T2 induced mycotoxicosis, in particular to protect against a decrease in average daily weight gain, intestinal damage, skin damage and snout damage, thus one or more of these signs of mycotoxicosis induced by T2 as a result of the ingestion of T2.

### BACKGROUND OF THE INVENTION

The invention in general pertains to protection against mycotoxicosis induced by mycotoxins. In particular, the invention pertains to protection against mycotoxicosis induced by T-2 toxin (type A trichothecenes-2 toxin or T2).

Mycotoxins in general are highly diverse secondary metabolites produced in nature by a wide variety of fungus which causes food contamination, resulting in mycotoxicosis in animals and humans. In particular, trichothecenes mycotoxin produced by genus Fusarium is agriculturally more important worldwide due to the potential health hazards they pose. It is mainly metabolized and eliminated after ingestion, yielding more than 20 metabolites with the hydroxy trichothecenes-2 toxin being the major metabolite. Trichothecene is hazardously intoxicating due to their additional potential to be topically absorbed, and their metabolites affect the gastrointestinal tract, skin, kidney, liver, and immune and hematopoietic progenitor cellular systems. Sensitivity to this type of toxin varying from dairy cattle to pigs, with the most sensitive endpoints being neural, reproductive, immunological and hematological effects. The mechanism of action mainly consists of the inhibition of protein synthesis and oxidative damage to cells followed by the disruption of nucleic acid synthesis and ensuing apoptosis. The possible hazards, historical significance, toxicokinetics, and the genotoxic and cytotoxic effects along with regulatory guidelines and recommendations pertaining to the trichothecene mycotoxin are commonly known.

T-2 toxins are predominantly found in grains, such as wheat, maize, barley, rice, soybeans and particularly in oats and products thereof. The fungal propagation and production of T-2 is enhanced in developing countries around the world due to tropical conditions like high temperatures and moisture levels, monsoons, unseasonal rains during harvests and flash floods. The production of T-2 is enhanced by factors such as the humidity of the substrate, the relative humidity, the temperature and the availability of oxygen.

T-2 is readily absorbed by various modes, including the topical, oral, and inhalational routes. As a dermal irritant and blistering agent, it is alleged to be 400 times more intoxicating than sulfur mustard. Respiratory ingestion of the toxin indicates its activity being comparable to that of mustard or lewisite. The T-2 mycotoxin is distinctive in that systemic toxicity can result from any route of exposure, i.e., dermal, oral, or respiratory.

The toxicity and deleterious effects of T-2 vary on the basis of numerous factors, such as the route of administration; the time and amount of exposure; the dosage administered; and the age, sex and overall health of the animal along with presence of any other mycotoxin. Intoxication often occurs after feeding on feed made from grain, hay and straw, wintering in the open and becoming contaminated with F. sporotrichiella and F. poae. Illustrative symptoms of T-2 induced mycotoxicosis are emesis, vomiting, skin blistering, loss of appetite and weight loss.

Ruminants are known to be relatively resistant to the T-2 toxin in comparison to monogastric animals. In poultry, the T-2 toxin has been the causative agent for mouth and intestinal lesions in addition to the impairment of immune responses, destruction of the hematopoietic system, declining egg production, the thinning of egg shells, refusal of feed, weight loss and altered feather patterns, abnormal positioning of the wings, hysteroid seizures or an impaired righting reflex [49, 50]. It has been reported that poultry are relatively less susceptible to trichothecenes than pigs. In pigs, along with serous-haemorrhagic necrotic-ulcerative inflammation of the digestive tract, some necrosis are established on the snout, lips and tongue, edema and mucous coatings of the mucosa of the stomach, swelling in the region of the head, especially around the eyelids and larynx, and sometimes even paresis or paralysis are seen. Toxic effects of the T-2 toxin are usually manifested in the form of alimentary toxic aleukia (ATA). The symptoms include vomiting, diarrhea, leukopenia, hemorrhage, shock and death. Acute toxicological effects are also characterized by multiple hemorrhages of the serosa of the liver and along the intestinal tract, stomach and esophagus.

Indeed, the prospects of the trichothecene as potential hazardous agents, decontamination strategies and future perspectives are comprehensively described in the art. Regarding treatment against T-2 induced mycotoxicosis, this is mainly restricted to detection strategies pertaining to maximum permissible limits in feed and food stocks. Still, its presence can prove to be toxic. Presently, T-2 toxin treatments of induced damage emphasize mainly the use of natural substances, probiotics, and amino acids, and the quest for a precise antidote against the toxin continues to date. Therefore, stringent regulations are established and quarantine activities are undertaken in order to prevent unplanned exposure on a large scale. Although it has been mentioned (see e.g. Manohar V. et al., “Final Report: Development Of Vaccines To The Mycotoxin T-2”, Borriston Laboratories, Maryland, USA, 15 Mar. 1985, AD-A158 544/7/XAB 16p, NTIS database) that subject animals can be vaccinated against T-2, this has been done consistently using the strategy of ant-idiotypic vaccination, where an antibody response is elicited against T-2 specific antibodies. This strategy however has not been found to be successful in prophylactic treatment of T2 induced mycotoxicis. Therefore, prophylactic treatment of T2 induced mycotoxicosis is currently mainly restricted to good agricultural practice to reduce mycotoxins production on crop and control programs of food and feed commodities to ensure that mycotoxin levels remain below certain limits.

Fungi in general cause a broad range of diseases in animals, involving parasitism of organs and tissues as well as allergenic manifestations. However, other than poisoning through ingestion of non-edible mushrooms, fungi can produce mycotoxins and organic chemicals that are responsible for various toxic effects referred to as mycotoxicosis. This disease is caused by exposure to mycotoxins, pharmacologically active compounds produced by filamentous fungi contaminating foodstuffs or animal feeds. Mycotoxins are secondary metabolites not critical to fungal physiology, that are extremely toxic in minimum concentrations to vertebrates upon ingestion, inhalation or skin contact. About 400 mycotoxins are currently recognized, subdivided in families of chemically related molecules with similar biological and structural properties. Of these, approximately a dozen groups regularly receive attention as threats to animal health. Examples of mycotoxins of greatest public interest and agroeconomic significance include aflatoxins (AF), ochratoxins (OT), trichothecenes (T; including deoxynivalenol, abbreviated DON), zearalenone (ZEA), fumonisin (F), tremorgenic toxins, and ergot alkaloids. Mycotoxins have been related to acute and chronic diseases, with biological effects that vary mainly according to the diversity in their chemical structure, but also with regard to biological, nutritional and environmental factors. The pathophysiology of mycotoxicosis is the consequence of interactions of mycotoxins with functional molecules and organelles in the animal cell, which may result in carcinogenicity, genotoxicity, inhibition of protein synthesis, immunosuppression, dermal irritation, and other metabolic perturbations. In sensitive animal species, mycotoxins may elicit complicated and overlapping toxic effects. Mycotoxicosis are not contagious, nor is there significant stimulation of the immune system. Treatment with drugs or antibiotics has little or no effect on the course of the disease. To date no human or animal vaccine is available for combating mycotoxicosis.

A growing body of work is thus focusing in developing vaccines and/or immunotherapy with efficacy against broad fungal classes as a powerful tool in combating mycoses, i.e. the infection with the fungi as such, instead of the toxins, in the prevention of specific fungal diseases. In contrast to mycoses, mycotoxicosis do not need the involvement of the toxin producing fungus and are considered as abiotic hazards, although with biotic origin. In this sense, mycotoxicosis have been considered examples of poisoning by natural means, and protective strategies have essentially focused on exposure prevention. Human and animal exposure occurs mainly from ingestion of the mycotoxins in plant-based food. Metabolism of ingested mycotoxins could result in accumulation in different organs or tissues; mycotoxins can thus enter into the human food chain through animal meat, milk, or eggs (carry over). Because toxigenic fungi contaminate several kinds of crops for human and animal consumption, mycotoxins may be present in all kinds of raw agricultural materials, commodities and beverages. The Food and Agriculture Organization (FAO) estimated that 25% of the world's food crops are significantly contaminated with mycotoxins. At the moment, the best strategies for mycotoxicosis prevention include good agricultural practice to reduce mycotoxins production on crop and control programs of food and feed commodities to ensure that mycotoxin levels stand below predetermined threshold limits. These strategies may limit the problem of contamination of commodities with some groups of mycotoxins with high costs and variable effectiveness. Except for supportive therapy (e.g., diet, hydration), there are almost no treatments for mycotoxin exposure and antidotes for mycotoxins are generally not available, although in individual exposed to AFs some encouraging results have been obtained with some protective agents such as chlorophyllin, green tea polyphenols and dithiolethiones (oltipraz).

In the art, particular vaccination strategies have been proposed against some mycotoxins, mainly to prevent mycotoxicosis by contamination of important foods of animal origin with a strategy based on the production of antibodies that could specifically block initial absorption or bioactivation of mycotoxins, their toxicity and/or secretion in animal products (such as milk) by immuno-interception, directed mainly at preventing mycotoxicosis in humans.

The production of vaccines for protection against mycotoxicosis however are very challenging, principally related to the fact that the mycotoxins themselves are small non-immunogenic molecules, and the toxicity associated with mycotoxins which makes the use as antigens in healthy subjects not risk free. Mycotoxins are low molecular weight, usually non-proteinaceous molecules, which are not ordinarily immunogenic (haptens), but can potentially elicit an immune response when attached to a large carrier molecule such as a protein. Methods for conjugation of mycotoxins to protein or polypeptide carrier and optimization of conditions for animal immunization have been extensively studied, with the purpose of producing monoclonal or polyclonal antibodies with different specificities to be used in immunoassay for screening of mycotoxins in products destined for animal and human consumption. Coupling proteins used in these studies included bovine serum albumin (BSA), keyhole limpet haemocyanin (KLH), thyroglobulin (TG) and polylysine, among others. In the past decades, many efforts have been made for developing mycotoxin derivatives that can be bound to proteins while retaining enough of the original structure so that antibodies produced will recognize the native toxin. Through these methods, antibodies against many mycotoxins have been made available, demonstrating that conjugation to proteins may be an effective tool for the raise of antibodies. The application of this strategy for human and animal vaccination, thus, to arrive at protection while being safe for the recipient, has not been successful so far due to the toxic properties of the molecules that might be released in vivo. For example, conjugation of toxins such as T-2 to protein carriers has been shown to result in unstable complexes with potential release of the free toxin in its active form (Chanh et al, Monoclonal anti-idiotype induces protection against the cytotoxicity of the trichothecene mycotoxin T-2, in J Immunol. 1990, 144: 4721-4728). In analogy with toxoid vaccines, which may confer a state of protection against the pathological effects of bacterial toxins, a reasonable approach to the development of vaccines against mycotoxin may be based on conjugated “mycotoxoids”, defined as modified form of mycotoxins, devoid of toxicity although maintaining antigenicity (Giovati L et al, Anaflatoxin B1 as the paradigm of a new class of vaccines based on “Mycotoxoids”, in Ann Vaccines Immunization 2(1): 1010, 2015). Given the non-proteinaceous nature of mycotoxins, the approach for conversion to mycotoxoids should rely on chemical derivatization. The introduction of specific groups in strategic positions of the related parent mycotoxin may lead to formation of molecules with different physicochemical characteristics, but still able to induce antibodies with sufficient cross-reacting to the native toxin. The common rationale for mycotoxin vaccination would thus be based on generating antibodies against the mycotoxoid with an enhanced ability to bind native mycotoxin compared with cellular targets, neutralizing the toxin and preventing disease development in the event of exposure. A potential application of this strategy has been demonstrated in the case of mycotoxins belonging to the AF group (Giovati et al, 2015), but not for any of the other mycotoxins. Moreover, the protective effect has not been demonstrated against mycotoxicosis of the vaccinated animal as such, but only against carry over in dairy cows to their milk, so as to protect people that consume the milk or products made thereof from mycotoxicosis.

### OBJECT OF THE INVENTION

It is an object of the invention to provide a method to protect an animal against mycotoxicosis induced by T-2 toxin, an important mycotoxin in animal feed.

### SUMMARY OF THE INVENTION

In order to meet the object of the invention it has been found that conjugated T-2 toxin (T2) is suitable for use in a method to protect an animal against T2 induced mycotoxicosis. It was found that there was no need to convert the T2 into a toxoid, the conjugated toxin appeared to be safe for the treated host animal. Also, it was surprising to see that an immune response induced against a small molecule such as a mycotoxin is, is strong enough to protect the animal itself against mycotoxicosis after ingestion of the mycotoxin post treatment. Such actual protection of an animal by inducing in that animal an immune response against a mycotoxin itself has not been shown in the art for any mycotoxin.

### Definitions

Mycotoxicosis is the disease resulting from exposure to a mycotoxin. The clinical signs, target organs, and outcome depend on the intrinsic toxic features of the mycotoxin and the quantity and length of exposure, as well as the health status of the exposed animal.

To protect against mycotoxicosis means to prevent or decrease one or more of the negative physiological effects of the mycotoxin in the animal, such as a decrease in average daily weight gain, intestinal damage, skin damage and snout damage.

T-2 toxins (also denoted as T-2 mycotoxin, T-2 fusariotoxin, Insariotoxin or Trichothecene) are the mycotoxins that have a tetracyclic sesquiterpenoid 12,13-epoxytrichothec-9-ene ring in common, which epoxy ring is responsible for the toxicological activity. Their chemical structure is characterized by hydroxyl group at the C-3 position, acetyloxy groups at the C-4 and C-15 positions, hydrogen at the C-7 position, and an ester-linked isovaleryl group at the C-8 position (instead of a carbonyl group for other types of trichothecenes such as deoxynivalenol), as indicated in formula 1 here below:

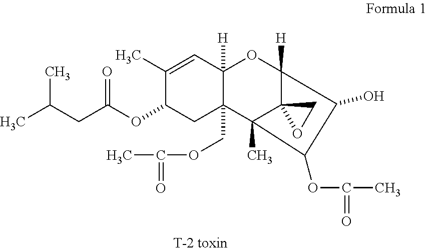 [SMILES: CC(=O)OC[C@]12C[C@H](OC(=O)CC(C)C)C(C)=C[C@H]1O[C@@H]1[C@H](O)C(OC(C)=O)[C@@]2(C)[C@]12CO2]

A conjugated molecule is a molecule to which an immunogenic compound is coupled through a covalent bond. Typically the immunogenic compound is a large protein such as KLH, BSA or OVA.

An adjuvant is a non-specific immunostimulating agent. In principal, each substance that is able to favor or amplify a particular process in the cascade of immunological events, ultimately leading to a better immunological response (i.e. the integrated bodily response to an antigen, in particular one mediated by lymphocytes and typically involving recognition of antigens by specific antibodies or previously sensitized lymphocytes), can be defined as an adjuvant. An adjuvant is in general not required for the said particular process to occur, but merely favors or amplifies the said process. Adjuvants in general can be classified according to the immunological events they induce. The first class, comprising i.a. ISCOM's (immunostimulating complexes), saponins (or fractions and derivatives thereof such as Quil A), aluminum hydroxide, liposomes, cochleates, polylactic/glycolic acid, facilitates the antigen uptake, transport and presentation by APC's (antigen presenting cells). The second class, comprising i.a. oil emulsions (either W/O, O/W, W/O/W or O/W/O), gels, polymer microspheres (Carbopol), non-ionic block copolymers and most probably also aluminum hydroxide, provide for a depot effect. The third class, comprising i.a. CpG-rich motifs, monophosphoryl lipid A, mycobacteria (muramyl dipeptide), yeast extracts, cholera toxin, is based on the recognition of conserved microbial structures, so called pathogen associated microbial patterns (PAMPs), defined as signal 0. The fourth class, comprising i.a. oil emulsion surface active agents, aluminum hydroxide, hypoxia, is based on stimulating the distinguishing capacity of the immune system between dangerous and harmless (which need not be the same as self and non-self). The fifth class, comprising i.a. cytokines, is based on upregulation of costimulatory molecules, signal 2, on APCs.

A vaccine is in the sense of this invention is a constitution suitable for application to an animal, comprising one or more antigens in an immunologically effective amount (i.e. capable of stimulating the immune system of the target animal sufficiently to at least reduce the negative effects of a challenge with a disease inducing agent, typically combined with a pharmaceutically acceptable carrier (i.e. a biocompatible medium, viz. a medium that after administration does not induce significant adverse reactions in the subject animal, capable of presenting the antigen to the immune system of the host animal after administration of the vaccine) such as a liquid containing water and/or any other biocompatible solvent or a solid carrier such as commonly used to obtain freeze-dried vaccines (based on sugars and/or proteins), optionally comprising immunostimulating agents (adjuvants), which upon administration to the animal induces an immune response for treating a disease or disorder, i.e. aiding in preventing, ameliorating or curing the disease or disorder.

### Further Embodiments of the Invention

In a further embodiment of the invention, the conjugated T2 is systemically administered to the animal. Although local administration, for example via mucosal tissue in the gastro-intestinal tract (oral or anal cavity) or in the eyes (for example when immunising chickens) is known to be an effective route to induce an immune response in various animals, it was found that systemic administration leads to an adequate immune response for protecting animals against a T2 induced mycotoxicosis. It was found in particular that effective immunisation can be obtained upon intramuscular, oral and/or intradermal administration.

The age of administration is not critical, although it is preferred that the administration takes place before the animal is able to ingest feed contaminated with substantial amounts of T2. Hence a preferred age at the time of administration of 6 weeks or younger. Further preferred is an age of 4 weeks or younger, such as for example an age of 1-3 weeks.

In yet another embodiment of the invention the conjugated T2 is administered to the animal at least twice. Although many animals (in particular swine chickens, ruminants) in general are susceptible for immunisation by only one shot of an immunogenic composition, it is believed that for economic viable protection against T2 two shots are preferred. This is because in practice the immune system of the animals will not be triggered to produce anti-T2 antibodies by natural exposure to T2, simply because naturally occurring T2 is not immunogenic. So, the immune system of the animals is completely dependent on the administration of the conjugated T2. The time between the two shots of the conjugated T2 can be anything between 1 week and 1-2 years. For young animals it is believed that a regime of a prime immunisation, for example at 1-3 weeks of age, followed by a booster administration 1-4 weeks later, typically 1-3 weeks later, such as 2 weeks later, will suffice. Older animals may need a booster administration every few months (such as 4, 5, 6 months after the last administration), or on a yearly or biannual basis as is known form other commercially applied immunisation regimes for animals.

In still another embodiment the conjugated T2 is used in a composition comprising an adjuvant in addition to the conjugated T2. An adjuvant may be used if the conjugate on itself is not able to induce an immune response to obtain a predetermined level of protection. Although conjugate molecules are known that are able to sufficiently stimulate the immune system without an additional adjuvant, such as KLH or BSA, it may be advantageous to use an additional adjuvant. This could take away the need for a booster administration or prolong the interval for the administration thereof. All depends on the level of protection needed in a specific situation. A type of adjuvant that was shown to be able and induce a good immune response against T2 when using conjugated-T2 as immunogen is an emulsion of water and oil, such as for example a water-in-oil emulsion or an oil-in-water emulsion. The former is typically used in poultry while the latter is typically used in animals who are more prone to adjuvant induced site reactions such as swine and ruminants.

In again another embodiment the conjugated T2 comprises T2 conjugated to a protein having a molecular mass above 10.000 Da. Such proteins, in particular keyhole limpet hemocyanin (KLH) and ovalbumin (OVA), have been found to be able and induce an adequate immune response in animals, in particular in swine and chickens. A practical upper limit for the protein might be 100 MDa.

Regarding the protection against mycotoxicosis, it was found in particular that using the invention, the animal is believed to be protected against a decrease in average daily weight gain, liver damage and damage to the intestinal tract, in particular the stomach, thus one or more of these signs of mycotoxicosis induced by T2.

The invention will now be further explained using the following examples.

### EXAMPLES OF THE INVENTION

In a first series of experiments (see Examples 1-4) it was assessed whether an active immune response against a mycotoxin can be elicited using a conjugated mycotoxin, and if so, is able to protect the vaccinated animal against a disorder induced by this mycotoxin after ingestion thereof. For the latter a pig model for challenge with DON was used. Thereafter (Example 5) it was assessed whether or not the use of conjugated T2 in a vaccine can induce antibodies against T-2 toxin in the vaccinated animal.

### Example 1: Immunisation Challenge Experiment Using Conjugated DON

### Objective

The objective of this study was to evaluate the efficacy of conjugated deoxynivalenol to protect an animal against mycotoxicosis due to DON ingestion. To examine this, pigs were immunised twice with DON-KLH before being challenged with toxic DON. Different routes of immunisation were used to study the influence of the route of administration.

### Study Design

Forty 1 week old pigs derived from 8 sows were used in the study, divided over 5 groups. Twenty-four piglets of group 1-3 were immunised twice at 1 and 3 weeks of age. Group 1 was immunised intramuscularly (IM) at both ages. Group 2 received an IM injection at one week of age and an oral boost at three weeks of age. Group 3 was immunised intradermally (ID) two times. From 5% weeks of age groups 1-3 were challenged during 4 weeks with DON administered orally in a liquid. Group 4 was not immunised but was only challenged with DON as described for groups 1-3. Group 5 served as a control and only received a control fluid, from the age of 5.5 weeks for 4 weeks.

The DON concentration in the liquid formulation corresponded to an amount of 5.4 mg/kg feed. This corresponds to an average amount of 2.5 mg DON per day. After four weeks of challenge all animals were post-mortem investigated, with special attentions for the liver, kidneys and the stomach. In addition, blood sampling was done at day 0, 34, 41, 49, 55, 64 (after euthanasia) of the study, except for group 5 of which blood samples were taken only at day 0, 34, 49, and directly after euthanasia.

### Test Articles

Three different immunogenic compositions were formulated, namely Test Article 1 comprising DON-KLH at 50 μg/ml in an oil-in-water emulsion for injection (X-solve 50, MSD AH, Boxmeer) which was used for IM immunization; Test Article 2 comprising DON-KLH at 50 μg/ml in a water-in-oil emulsion (GNE, MSD AH, Boxmeer) which was used for oral immunization and Test Article 3 comprising DON-KLH at 500 μg/ml in an oil-in-water emulsion for injection (X-solve 50) for ID immunisation.

The challenge deoxynivalenol (obtained from Fermentek, Israel) was diluted in 100% methanol at a final concentration of 100 mg/ml and stored at <−15° C. Prior to usage, DON was further diluted and supplied in a treat for administration.

### Inclusion Criteria

Only healthy animals were used. In order to exclude unhealthy animals, all animals were examined before the start of the study for their general physical appearance and absence of clinical abnormalities or disease. Per group piglets from different sows were used. In everyday practice all animals will be immunised even when pre-exposed to DON via intake of DON contaminated feed. Since DON as such does not raise an immune response, it is believed that there is no principle difference between animals pre-exposed to DON and naïve with respect to DON.

### Results

None of the animals had negative effects associated with the immunisation with DON-KLH. The composition thus appeared to be safe.

All pigs were serologically negative for titres against DON at the start of the experiment, During the challenge the groups immunised intramuscular (Group 1) and intradermally (Group 3) developed antibody responses against DON as measured by ELISA with native DON-BSA as the coating antigen. Table 1 depicts the average IgG values on 4 time points during the study with their SD values. Both Intramuscular immunisation and Intradermal immunisation induced significant titres against DON.

TABLE 1
IgG titres

| | group 1 | group 2 | group 3 | group 4 | Group 5 |
|---|---|---|---|---|---|
| T = 0 | <4.3 | <4.3 | <4.3 | <4.3 | <4.3 |
| T = 35 | 11.2 | 4.86 | 9.99 | 4.3 | 4.19 |
| T = 49 | 9.56 | 4.64 | 8.81 | 4.71 | 3.97 |
| T = 64 | 8.48 | 4.3 | 7.56 | 4.3 | 3.31 |

As depicted in Table 2 all immunised animals, including the animals in Group 2 that showed no significant anti-DON IgG titre increase, showed a significant higher weight gain during the first 15 days compared to the challenge animals. With respect to the challenged animals, all animals gained more weight over the course of the study.

TABLE 2
weight analysis

| | | | | | Average additional |
|---|---|---|---|---|---|
| | | | | | weight gain compared |
| | | | weight | weight | to challenge animals |
| | ADG1¹ | ADG² | begin | end | (grams) |
| group 1 | 0.67 | 0.80 | 11.63 | 32.29 | +1060 |
| group 2 | 0.64 | 0.79 | 12.31 | 32.13 | +760 |
| group 3 | 0.58 | 0.82 | 12.88 | 32.25 | +310 |
| group 4 | 0.54 | 0.81 | 12.69 | 31.75 | 0 |
| group 5 | 0.57 | 0.80 | 11.63 | 31.08 | +390 |

¹average daily weight gain over the first 15 days of the challenge
²average daily weight gain over the last 13 days of the challenge

The condition of the small intestines (as determined by the villus/crypt ratio in the jejunum) was also monitored. In table 3 the villus/crypt ratio is depicted. As can be seen, the animals in group 3 had an average villus crypt/crypt ratio comparable to the healthy controls (group 5), while the non-immunised, challenged group (group 4) had a much lower (statistically significant) villus crypt ratio. In addition, group 1 and group 2, had a villus/crypt ratio which was significantly better (i.e. higher) compared to the non-immunised challenge control group. This indicates that the immunisation protects against the damage of the intestine, initiated by DON.

TABLE 3
villus/crypt ratio

| | group 1 | group 2 | group 3 | group 4 | group 5 |
|---|---|---|---|---|---|
| average | 1.57 | 1.41 | 1.78 | 1.09 | 1.71 |
| STD | 0.24 | 0.22 | 0.12 | 0.10 | 0.23 |

The general condition of other organs was also monitored, more specifically the liver, the kidneys and the stomach. It was observed that all three test groups (groups 1-3) were in better health than the non-immunised challenge control group (group 4). In table 4 a summary of the general health data is depicted. The degree of stomach ulcer is reported from − (no prove of ulcer formation) to ++ (multiple ulcers). The degree of stomach inflammation is reported from − (no prove of inflammation) to ++/−(initiation of stomach inflammation).

TABLE 4
General health data

| | | | Stomach | |
|---|---|---|---|---|
| | Liver colour | Stomach ulcer | inflammation | Kidneys |
| Group 1 | Normal-yellow | − | − | Pail |
| Group 2 | Normal | +/−− | − | Normal |
| Group 3 | Normal | +/− | +/−− | Normal |
| Group 4 | Pail | ++ | ++/− | Pail |
| Group 5 | Normal | + | ++/− | Normal |

### Example 2: Effect of Immunisation on DON Levels

### Objective

The objective of this study was to evaluate the effects of immunization with a DON conjugate on the toxicokinetics of DON ingestion. To examine this, pigs were immunised twice with DON-KLH before being fed toxic DON.

### Study Design

Ten 3 week old pigs were used in the study, divided over 2 groups of 5 pigs each. The pigs in Group 1 were immunised IM twice at 3 and 6 weeks of age with DON-KLH (Test Article 1; example 1). Group 2 served as a control and only received a control fluid. At the age of 11 weeks the animals were each administered DON (Fermentek, Israel) via a bolus at a dose of 0.05 mg/kg which (based on the daily feed intake) resembled a contamination level of 1 mg/kg feed. Blood samples of the pigs were taken juts before DON administration and 0.25, 0.5, 0.75, 1, 1.5, 2, 3, 4, 6, 8, and 12 h post DON administration.

### Inclusion Criteria

Only healthy animals were used.

### Analysis of DON in Plasma

Plasma analysis of unbound DON was done using a validated LC-MS/MS method on an Acquity® UPLC system coupled to a Xevo® TQ-S MS instrument (Waters, Zellik, Belgium). The lower limit of quantification of DON in pig plasma using this method is 0.1 ng/ml.

### Toxicokinetic Analysis

Toxicokinetic modeling of the plasma concentration-time profiles of DON was done by noncompartmental analysis (Phoenix, Pharsight Corporation, USA). Following parameters were calculated: area under the curve from time zero to infinite (AUC_{0→∞}), maximal plasma concentration (Cₘₐₓ), and time at maximal plasma concentration (tₘₐₓ).

### Results

The toxicokinetic results are indicated in table 5 here beneath. As can be seen immunisation with DON-KLH decreases all toxicokinetic parameters. As it is unbound DON that is responsible for the exertion of toxic effects, it may be concluded that immunisation with DON-KLH will reduce the toxic effects caused by DON by reducing the amount of unbound DON in the blood of animals.

TABLE 5
Toxicokinetic parameters of unbound DON

| Toxicokinetic parameter | DON-KLH | Control |
|---|---|---|
| AUC_{0→∞} | 77.3 ± 23.6 | 187 ± 33 |
| Cₘₐₓ | 12.5 ± 2.7 | 30.8 ± 2.5 |
| tₘₐₓ | 1.69 ± 1.03 | 2.19 ± 1.07 |

### Example 3: Serological Response Against Various DON Conjugates

### Objective

The objective of this study was to evaluate the efficacy of different conjugated deoxynivalenol products.

### Study Design

Eighteen 3 week old pigs were used in the study, divided over 3 groups of six pigs each. The pigs of group 1 were immunised twice intramuscularly at 3 and 5 weeks of age with DON-KLH (using Test Article 1 of Example 1). Group 2 was immunised correspondingly with DON-OVA. Group 3 served as a negative control. All animals were checked for an anti-DON IgG response at 3 weeks of age, 5 weeks of age and 8 weeks of age.

### Results

The serological results are indicated here below in the table in log 2 antibody titre.

TABLE 6
anti-DON IgG response

| Test Article | 3 weeks | 5 weeks | 8 weeks |
|---|---|---|---|
| DON-KLH | 3.5 | 6.6 | 8.3 |
| DON-OVA | 3.3 | 3.9 | 11.8 |
| Control | 4.8 | 3.3 | 3.3 |

It appears that both conjugates are suitable to raise an anti-DON IgG response. Also, a response appears be induced by one shot only.

### Example 4: Serological Response in Chickens

### Objective

The objective of this study was to evaluate the serological response of DON-KLH in chickens.

### Study Design

For this study 30 four week-old chickens were used, divided over three groups of 10 chickens each. The chickens were immunized intramuscularly with DON-KLH. Group 1 was used as a control and received PBS only. Group 2 received DON-KLH without any adjuvant and group 3 received DON-KLH formulated in GNE adjuvant (available from MSD Animal Health, Boxmeer). A prime immunization was given on day 0 with 0.5 ml vaccine into right leg. On day 14, chickens received a comparable booster immunization into the left leg.

Blood sampling took place at day 0 and 14, as well as on day 35, 56, 70 and 84. Serum was isolated for the determination of IgY against DON. At day 0 and 14 blood samples were isolated just before immunisation.

### Results

The serological results are depicted in table 7 in log 2 antibody titre. The PBS background has been subtracted from the data.

TABLE 7
anti-DON IgY response

| Vaccine | Day 0 | Day 14 | Day 35 | Day 56 | Day 70 | Day 84 |
|---|---|---|---|---|---|---|
| DON-KLH | 0 | 0 | 0.6 | 1.2 | 1.1 | 1.2 |
| DON-KLH | 0 | 1.9 | 6.5 | 6.0 | 6.7 | 7.7 |
| in GNE | | | | | | |

As can be seen, the conjugated DON also induces an anti-DON titre in chickens. GNE adjuvant increases the response substantially but appears to be not essential for obtaining a net response as such.

### Example 5: Serological Response Against T2 Conjugate

### Objective

The aim of this experiment was to assess whether or not the use of conjugated T2 in a vaccine can induce antibodies against T-2 toxin in the vaccinated animal.

### Study Design

For this a vaccine comprising T-2 toxin conjugated to Keyhole limpet hemocyanin (T2-KLH) was used. The conjugate was mixed with an oil-in water emulsion adjuvant (XSolve 50, MSD Animal Health, The Netherlands) at a final concentration of 115 μg/ml for intramuscular (IM) administration, or 1150 μg/ml for intradermal (ID) administration.

In the experiment also a DON vaccine as described here above was used as a positive control. Next to this, vaccines with other conjugated mycotoxins were formulated and used. In particular, zearalenone (ZEA) conjugated to Keyhole limpet hemocyanin (ZEA-KLH) and fumonisin (FUM) conjugated to KLH (T2-KLH) were formulated into vaccines. The conjugates were mixed with the oil-in water emulsion adjuvant (XSolve) as mentioned here above at a final concentration of 50 μg/ml for intramuscular (IM) administration or 500 μg/ml for intradermal (ID) administration respectively.

In the experiment 6 groups of 5 animals were used for vaccination at three weeks of age, Group 1 received 0.2 ml of FUM-KLH twice Intradermal, Group 2 received 0.2 ml ZEA-KLH twice, Group 3 was vaccinated with 2.0 ml DON-KLH IM in X-Solve 50 twice, Group 4 received 2.0 ml FUM-KLH IM twice, Group 5 received 2.0 ml ZEA-KLH twice IM, and finally Group 6 was vaccinated with 2.0 ml T2-KLH IM twice. There was a control group of three piglets, which control group received no vaccination. All primes were at three weeks of age and the boosters were at five weeks of age. The animals were monitored for 14 weeks after start of the study.

### Results

All pigs were serologically negative for titres against FUM, ZEA, T2 and DON at the start of the experiment, and all vaccinated groups developed antibody titres. The resulting log 2 titres are presented in Table 8 below. As can be seen, antibodies could be raised at high levels against each of the conjugated mycotoxins. This supports that the vaccine can be effectively used against the corresponding mycotoxicosis, as shown here above for DON induced mycotoxicosis.

TABLE 8
IgG titres

| | T = | T = | T = | T = | T = | T = | T = |
|---|---|---|---|---|---|---|---|
| Group | 0 | 28 | 42 | 56 | 70 | 84 | 91 |
| 1 | <3.3 | 12.2 | 11.1 | 9.9 | 8.5 | 7.1 | 6.7 |
| 2 | <4.3 | 10.1 | 8.8 | 8.6 | 6.7 | 6.0 | 5.4 |
| 3 | <4.3 | 10.5 | 9.5 | 8.5 | 7.6 | 6.5 | 6.6 |
| 4 | <3.3 | 15.4 | 14.7 | 13.1 | 12.6 | 10.6 | 10.1 |
| 5 | <4.3 | 12 | 10.9 | 11.5 | 8.8 | 8.1 | 8.0 |
| 6 | <3.3 | 13.5 | 12.6 | 11.4 | 10.3 | 9.1 | 8.9 |
| control FUM | <3.3 | <3.3 | <3.3 | <3.3 | <3.3 | <3.3 | <3.3 |
| control ZEA | <4.3 | <4.3 | <4.3 | <4.3 | <4.3 | <4.3 | <4.3 |
| control T2 | <3.3 | <3.3 | <3.3 | <3.3 | <3.3 | <3.3 | <3.3 |
| control DON | <4.3 | <4.3 | <4.3 | <4.3 | <4.3 | <4.3 | <4.3 |

### Example 6: Response Against T2 Conjugate in Chickens

### Objective

The aim of this experiment was to assess whether or not the use of conjugated T2 in a vaccine can induce protective antibodies against T2 in chickens.

### Study Design

For this a vaccine comprising T2 conjugated to Keyhole limpet hemocyanin (T2-KLH) was used in line with example 5. The conjugate was mixed with the oil emulsion adjuvant using the same mineral oil as used in example 5, and as an alternative in a comparable emulsion of a non-mineral oil, both at a final concentration of 50 μg/ml.

A group of 15 chickens were used in the study. Three groups of 5 animals were used. Group 1 was used as a negative control and was administered a PBS solution, Group 2 was vaccinated with T2-KLH mixed in the mineral oil containing adjuvant and Group 3 was vaccinated with the non-mineral oil containing adjuvant. The chickens were vaccinated intramuscularly with 0.5 ml of the vaccines at T=8 And T=22 (birds were included in the study at T=0 for acclimatization).

### Results

All chickens were serologically negative for titres against T2 at the start of the experiment (T=0, data not shown), and all vaccinated groups developed antibody titres. The resulting log 2 titres are presented in Table 9 below. As can be seen, antibodies could be raised at high levels against the conjugated T2 in both groups, although the induction of antibodies using the non-mineral oil seemed to be better. This supports the common understanding that the type of adjuvant is not essential for raising an adequate immune response as such, but the actual level of increasing the immune response may be adjuvant dependent.

TABLE 9
Antibody titres against T2 in chickens

| Group | T = 8 | T = 22 | T = 36 | T = 50 | T = 71 |
|---|---|---|---|---|---|
| 1 PBS | <3.1 | <3.1 | 4.0 | 5.0 | 5.2 |
| 2 T2-KLH mineral oil | 4.5 | 5.0 | 10.3 | 10.9 | 10.1 |
| 3 T2-KLH non mineral oil | 5.5 | 11.5 | 16.9 | 16.3 | 14.8 |

The serum samples from this study were additionally tested in an in vitro potency assay, were cells, (Caucasian colon adenocarcinoma cells), were incubated with the toxin alone, the toxin in combination with serum from a pool of positive animals in the ELISA and with the toxin in combination with serum from the PBS-injected (negative animals). The viability of the cell was measured by adding CCK8 and reading the optical density at 450 nm, table 10 depicts the results.

It can be observed that when comparing the positive sera in group one and two the OD450 values (viability of the cells) is increased compared to the negative serum in the same dilution (2× or 4×). Also, the OD increased when compared to adding no serum in combination with the T2. This indicates that the serum of the positive (vaccinated animals) is able to at least partly neutralize the effect of the toxin. Since negative serum cannot, this indicates the protective effect of the vaccine induced immune response.

TABLE 10
Neutralization data of chicken IgY on cells

| | 5 ng/ml T2 | 2.5 ng/ml T2 | no T-2 |
|---|---|---|---|
| 2× non-mineral oil adjuvant | 1.785 | 1.94 | 1.868 |
| 4× non-mineral oil adjuvant | 1.673 | 2.018 | 2.02 |
| no serum non-mineral oil test | 1.42 | 1.852 | 3.387 |
| 2× mineral oil adjuvant | 1.383 | 1.67 | 2.103 |
| 4× mineral oil adjuvant | 1.275 | 1.671 | 1.964 |
| no serum mineral oil test | 1.635 | 1.742 | 3.558 |
| 2× neg serum | 0.901 | 1.043 | 1.393 |
| 4× neg serum | 1.154 | 1.453 | 1.832 |
| no serum negative serum test | 1.633 | 1.931 | 3.388 |

### Example 7: Protection Against T2 Challenge in Pigs

### Objective

The aim of this experiment was to assess whether or not the use of conjugated T2 in a vaccine can induce protection against T2 challenge in pigs

### Study Design

For this the same vaccines comprising T2 conjugated to Keyhole limpet hemocyanin (T2-KLH) in two different adjuvants were used, one based on a mineral oil and the other based on a non-mineral oil as described in example 6. In the study a group of 24 pigs was used. A first group of 8 piglets were vaccinated with T2-KLH, albeit that a first subgroup of 4 animals received the vaccine based on the mineral oil containing adjuvant, and the second subgroup received the alternative vaccine. Both vaccines were administered intramuscularly in an amount of 2 ml at a concentration of 50 μg/ml. The animals were prime vaccinated at an age of 7-12 days (T=0), and booster vaccinated at an age of 21-26 days of age (T=14). Group 2 was not vaccinated but was challenged with T2 and served as a positive control. Group 3 was not vaccinated and not challenged and served as a negative control. The 16 challenged piglets of (groups 1 and 2) received at approximately 5.5 weeks of age 1.15 mg/kg feed of T2 daily for four weeks (0.56 mg/day) in a liquid formulation: the pigs received in the first week 0.19 mg T2/day in 16 ml fluid, in week 2 0.39 mg/day in 32 ml fluid, in week 3 0.72 mg/day in 45 ml of fluid and in week 4, 0.93 mg T2 per day in 60 ml fluid. Antibody titers were monitored over time. At the end of the study, the intestines, the skin and the snout of the piglets were evaluated.

### Results

All piglets were serologically negative for titres against T2 at the start of the experiment. During the challenge the vaccinated with T2-KLH developed antibody responses against T2, as depicted in Table 11, which shows the IgG values on 6 timepoints during the study.

TABLE 11
IgG titres against T2 in pigs

| | T = | T = | T = | T = | T = | T = |
|---|---|---|---|---|---|---|
| Group | 0 | 28 | 33 | 40 | 47 | 55 |
| 1a T2-KLH mineral oil | <3.3 | 14.2 | 14.0 | 13.1 | 12.4 | 11.5 |
| 1b T2-KLH non-mineral | <3.3 | 14.7 | 14.4 | 13.2 | 12.8 | 12.0 |
| 2 Positive control | <3.3 | <3.3 | <3.3 | <3.3 | <3.3 | <3.3 |
| 3 Negative control | <3.3 | <3.3 | <3.3 | <3.3 | <3.3 | <3.3 |

For all animals, the percentage of growth per piglet compared to the start weight at time of challenge was determined. The vaccination did not negatively impact growth. On the contrary, there was a slight increase in growth when comparing the vaccinated animals to the challenged animals. Moreover, vaccinated animals showed a better health status when looking at the intestines, the skin and the snout of the piglets.

Table 12 depicts the percentage of animals per group with the % weight gain during the challenge from the start weight of the challenge, moreover the % of animals with damage to a specific organ is depicted. This all shows that the conjugated T2 can be successfully used in a method to protect an animal against T2 induced mycotoxicosis.

TABLE 12
Weight and organ scores of piglets

| Group | weight gain | jejunum damage | skin damage | snout damage |
|---|---|---|---|---|
| 1a | 304% | 25 | 50 | 25 |
| 1b | 300% | 75 | 0 | 25 |
| 2 | 299% | 87.5 | 50 | 50 |
| 3 | 306% | 12.5 | 0 | 0 |

The improved intestinal health was confirmed with a higher (healthier) villus/crypt ratio in the vaccinated animals compared to the challenged animals, as depicted in Table 13.

TABLE 13
villus/crypt ratio

| Group | Villus/crypt ratio |
|---|---|
| Healthy controls | 1.67 |
| T2 challenge | 1.48 |
| T2 vaccination plus challenge | 1.79 |

1. A method of protecting an animal against T-2 toxin (T2)-induced mycotoxicosis comprising administering to the animal a conjugated T2.

2. The method according to claim 1, wherein the method protects the animal against one or more of the clinical signs of the T2 induced mycotoxicosis, and wherein the clinical signs are chosen from the group consisting of decreased weight gain, intestinal damage, skin damage and snout damage.

3. The method according to claim 1, wherein the conjugated T2 is systemically administered to the animal.

4. The method according to claim 3, wherein the conjugated T2 is administered intramuscularly, orally and/or intradermally.

5. The method according to claim 1, wherein the conjugated T2 is administered to the animal at an age of 6 weeks or younger.

6. The method according to claim 5 wherein the conjugated T2 is administered to the animal at an age of 4 weeks or younger.

7. The method according to claim 6, wherein the conjugated T2 is administered to the animal at an age of 1-3 weeks.

8. The method according to claim 1, wherein the conjugated T2 is administered to the animal at least twice.

9. (canceled)

10. (canceled)

11. (canceled)

12. (canceled)

13. (canceled)

14. The method according to claim 1, characterised in that the animal is a swine or chicken.

15. A vaccine comprising conjugated T2, an adjuvant and a pharmaceutically acceptable carrier.

16. The vaccine of claim 15, wherein the adjuvant is an emulsion of water and oil.

17. The vaccine of claim 16, wherein the adjuvant is a water-in-oil emulsion or an oil-in-water emulsion.

18. The vaccine of claim 15, wherein the conjugated T2 comprises T2 conjugated to a protein having a molecular mass above 10.000 Da.

19. The vaccine of claim 15, wherein the conjugated T2 comprises T2 conjugated to keyhole limpet hemocyanin (KLH) or ovalbumin (OVA).
